(19) United States
(12) Patent Application Publication
(10) Pub. No.: US 2017/0266284 A1
(43) Pub. Date: Sep. 21, 2017

(54) PERSONALIZED METHODS FOR TREATING DISEASE BY RADIOSENSITIZATION

(71) Applicants: Timothy J Kinsella, Providence, RI (US); Shuttle Pharmaceuticals, LLC, Rockville, MD (US)

(72) Inventors: Scott GRINDROD, Rockville, MD (US); Anatoly DRITSCHILO, Rockville, MD (US); Timothy J KINSELLA, Providence, RI (US)

(21) Appl. No.: 15/512,496

(22) PCT Filed: Sep. 18, 2015
(86) PCT No.: PCT/US15/50860

§ 371 Date: Mar. 17, 2017

Related U.S. Application Data
(60) Provisional application No. 62/052,081, filed on Sep. 18, 2014.

(51) Int. Cl. A61K 41/00; A61K 31/513; A61K 39/395; A61N 5/10; A61K 33/24; A61K 31/4745; A61K 9/00; A61K 31/7068; A61K 31/28

(52) U.S. Cl. CPC A61K 41/0038; A61K 31/7068; A61K 31/513; A61K 39/3955; A61K 31/28; A61K 33/24; A61K 31/4745; A61K 9/0019; A61K 9/0053; A61N 5/10

(57) ABSTRACT

Methods are provided for treating cancer and neoplastic diseases where such methods include the administration of radiosensitizing agents.

### CROSS-REFERENCE TO RELATED APPLICATIONS

This international application claims the benefit of U.S. Provisional Application No. 62/052,081, filed Sep. 18, 2014, the entirety of which is incorporated herein by reference.

### FIELD OF THE INVENTION

The present invention relates generally to methods of using radiosensitizing agents for treating disease and more particularly, but not exclusively, to methods of treating cancer by administering a halogenated nucleoside radiosensitizing agent in combination with radiation and additional chemotherapeutic agents.

### BACKGROUND OF THE INVENTION

Patients diagnosed with cancer can present with tumors having different or unique disease etiologies. To combat the unique presentation of cancer that may vary with each patient, personalized medicines and treatments have been developed to tailor medical treatment to the individual characteristics and needs of the patient. By identifying the distinctive disease characteristics present in the patient, a clinician can then provide a personalized therapy rather than resort to general treatments or therapies. New methods and treatment options are needed to identify distinct properties of certain cancers, to allow clinicians to exploit weaknesses in tumors and tumor cells and provide treatment options tailored to fit the patient's personal disease.

Of the weapons available in the treatment of cancer, radiation therapy is either a primary treatment or an adjuvant to surgery or chemotherapy. In principle, all cancers can be controlled if sufficient radiation doses can be delivered to tumor cells. In practice, the radiation dose is often limited by the toxicities associated with high doses of radiation on normal tissues. Moreover, many cancers can exhibit radio resistance that can limit the damage inflicted by known radiation therapy options. To preferentially deliver high doses of radiation to tumors, and limit doses to normal tissues, new methodologies are needed in the field.

A need exists for providing personalized, radiation-based therapies for treating cancer that overcome the radio-resistance offered by tumors and reduce the adverse effects of known irradiative therapies.

### SUMMARY OF THE INVENTION

The present invention meets the above-identified needs by providing methods for treating cancer that involve sensitizing cancer and/or neoplastic cells with a radiosensitizing agent, and then irradiating the tissues that include such cells with the aid of additional chemotherapeutic agents. The methods of the invention are applicable as personalized methods of treatment, specific to the patient, where the patient has first been tested for a distinguishing disease characteristic and such characteristic is incorporated into the design and deployment of the methods of the invention to treat the suspect disease (e.g., cancer).

In one aspect, the present invention includes a method for treating cancer in a patient in need of such treatment that includes administering a therapeutically effective amount of a radiosensitizing agent to the patient to sensitize cancerous cells to radiation. The radiosensitizing agent may comprise a halogenated nucleoside. The method may also include irradiating a selected tissue of the patient, where such tissue includes sensitized cancerous cells. Additionally, the method may include providing a therapeutically effective amount of a chemotherapeutic agent to the patient, which may enhance the radiation based therapy provided herein.

In another embodiment, the halogenated nucleosides may include a halogenated thymidine. The halogenated nucleoside may comprise 5-iodo-2-pyrimidinone-2′-deoxyribose (IPdR), 5-iodo-2′-doxyuridine (IUdR), 5-bromo-2′-deoxyuridine (BUdR), or a pharmaceutically acceptable salt thereof. In specific embodiments, the halogenated nucleoside may comprise 5-iodo-2-pyrimidinone-2′-deoxyribose (IPdR).

In a further embodiment, the cancers treated under the present methods of the invention may include pancreatic cancer, colorectal cancer, breast cancer, gastric cancer, metastatic breast cancer, head and neck cancers, endometrial cancer, ovarian cancer, ureter cancer, cervical cancer, esophageal cancer, bladder cancer, ovarian cancer, small-cell cancers, non-small cell cancers (e.g., non-small-cell lung cancer), malignant lymphomas, brain cancer, rectal cancer, sarcomas (e.g., soft tissue and/or bone sarcomas), or a combination thereof.

Moreover, the chemotherapeutic agents used in the practice of the invention may include 5-fluorouracil, bevacizumab, capecitabine, carboplatin, cisplatin, gemcitabine, irinotecan, oxaliplatin, mitomycin-c, paclitaxel, docetaxel, dacarbacine, tamoxifen, etoposide, pemetrexed, cyclophosphamide, doxorubicin, vincristine, amrubicin, an EGFR inhibitor, temozolomide, or a combination thereof.

In another aspect, the present invention includes a kit for implementing treatments of the invention in a patient in need of such treatment. The kit of the invention may include a radiosensitizing agent, a chemotherapeutic agent, and instructions for use of the radiosensitizing agent and the chemotherapeutic agent in combination with radiation therapy for treating cancer in the patient in need of such treatment.

In a further aspect, the present invention includes a method for treating a DNA mismatch repair (MMR) deficient cancer in a patient in need of such treatment, wherein cancer cells of the patient exhibit a substantial lack of MMR protein. The method may include the step of administering a radiosensitizing agent to the patient to sensitize the cancer cells to radiation. Moreover, the method may include irradiating the cancer cells that have been sensitized to radiation and administering at least one chemotherapeutic agent to the patient. Additionally, the method of the invention may provide for the administration of the radiosensitizing agent and at least one therapeutic agent in therapeutically effective amounts sufficient to sensitize the cancer cells to radiation or reduce the quantity of cancer cells or tumor burden in the patient.

In a still further aspect, the present invention includes a method for treating cancer in a patient in need of such treatment, wherein the cancer is selected from the group consisting of gastrointestinal cancer, gynecologic cancer, genitourinary cancer, non-small-cell lung cancer, and brain cancer. The method may comprise determining whether the patient has DNA mismatch repair (MMR) protein deficient cancer by retrieving at least one cancer cell from the patient and testing the at least one cancer cell to determine the level of MMR protein. Furthermore, the method may include administering to a patient determined to have MMR deficient cancer an amount of a radiosensitizing agent effective to sensitize cancerous cells to radiation. Moreover, the method may include the steps of (1) irradiating a selected tissue of the patient, wherein the selected tissue comprises sensitized, MMR deficient cancerous cells; and (2) providing a therapeutically effective amount of a chemotherapeutic agent to the patient.

The foregoing steps of the methods of the invention may be repeated as necessary to facilitate treatment of the specified disease.

### BRIEF DESCRIPTION OF THE DRAWINGS

The foregoing summary and the following detailed description of the exemplary embodiments of the present invention may be further understood when read in conjunction with the appended drawings, in which:

FIG. 1 diagrammatically illustrates the cellular processing of IPdR following oral administration and IPdR's role in radiation sensitization.

FIG. 2 diagrammatically illustrates a treatment method using the radiosensitizer IPdR in a patient having locally advanced rectal cancer.

FIG. 3 diagrammatically illustrates a specific treatment method using the radiosensitizer IPdR in a patient having a locally advanced sarcoma.

FIG. 4 diagrammatically illustrates a specific treatment method using the radiosensitizer IPdR in a patient having a deficient mismatch repair (MMR) pathway.

### DETAILED DESCRIPTION OF THE INVENTION

Radiation oncology has been driven by technological innovation; however, achieving a better dose distribution within the tumor volume has reached an effectiveness plateau and does not take advantage of biologic potential for improving therapeutic results.

The methods of the invention include treatments and therapies for treating cancer in a patient that may be in need of such treatment. Generally, the methods of the invention may include the administration of a radiosensitizing agent followed by the application radiation therapy. Radiation sensitizing agents may be defined as compounds that sensitize cancerous or neoplastic cells to radiation therapy. Moreover, the methods of the present invention may include the application of a chemotherapeutic agent in addition to the application of radiation therapy.

The term “neoplastic disease” according to the present invention refers to a proliferative disorder caused or characterized by the proliferation of cells, which are unrestrained by normal growth control. The term “cancer” according to the present invention includes benign and malignant tumors and any other proliferative disorders (e.g., the formation of metastasis). Cancers of the same tissue type in general originate from the same tissue, and are for example divided into different subtypes based on their biological characteristics. Specific examples of cancers that may be treated by the methods of the invention include solid tumors and may include pancreatic cancer, colorectal cancer, breast cancer, gastric cancer, non-small-cell lung cancer, metastatic breast cancer, head and neck cancers, endometrial cancer, ovarian cancer, ureter cancer, cervical cancer, esophageal cancer, bladder cancer, ovarian cancer, small-cell cancer and non-small cell cancer, malignant lymphomas, brain cancer (e.g, malignant glioma), sarcomas, and rectal cancer. In a preferred aspect, the methods of the invention pertain to treatments for brain cancer and rectal cancer.

As used herein, the term “mitotic index” refers to the number of cells in a tissue sample (e.g., biopsied tissue) that are undergoing mitoses per 100 counted cells. A “low mitotic index” refers to less than 4 cells undergoing mitosis per 100 counted cells. An “intermediate mitotic index” refers to between 4 and 10 cells undergoing mitosis per 100 counted cells. A “high mitotic index” refers to greater than 10 cells undergoing mitosis per 100 counted cells.

As used herein, the terms “administer,” “administration” or “administering” refer to (1) providing, giving, dosing, and/or prescribing by either a health practitioner or his authorized agent or under his or her direction according to the disclosure; and (2) putting into, taking or consuming by the patient or person himself or herself, according to the disclosure.

As used herein, the terms “treat,” “treatment,” and/or “treating” may refer to the management of a disease, disorder, or pathological condition (e.g., cancer or neoplastic disorder) with the intent to cure, ameliorate, stabilize, prevent, or control the disease, disorder, or pathological condition. Regarding control of the disease, disorder, or pathological condition more specifically, “control” may include the absence of disease progression, as assessed by the response to the methods recited herein, where such response may be complete (e.g., placing the disease in remission) or partial (e.g., slowing the spread of cancerous cells and tissues and/or preventing, slowing, or halting metastasis). For example, a patient responding to the methods of treatment disclosed in the present invention may exhibit the absence of disease progression (e.g., halting the growth and/or spread of neoplastic cells and tissues) over another patient that does not receive the methods of treatment described herein. Following treatment, if no detectable evidence of residual cancer is found in a tissue sample, the response to treatment may be considered a “pathologic complete response” or “pCR.”

In accordance with the invention, the methods may include the administration of a therapeutically effective amount of a radiosensitizing agent to a patient in order to sensitize neoplastic or cancerous cells and tissue to radiation. As used herein, the term “radiosensitizing agent” which may be read also as a “radiosensitizer” denotes an agent having an effect of enhancing the sensitivity of cancerous and/or neoplastic cells to radiation.

Preferably, the radiosensitizing agents of the invention include halogenated nucleosides and their analogs. For example, halogenated nucleoside analogs of the invention may include 5-iodo-2′-deoxyuridine (IUdR), 5-bromo-2′-dexoyuridine (BUdR), 5-iodo-2-pyrimidinone-2′-deoxyribose (IPdR), or a combination thereof. As set forth herein, the radiosensitizing agent of the invention may alternatively or additionally include one or more PdR analogs described in Cheng, et al., U.S. Pat. No. 5,728,684, the entirety of which is incorporated by reference herein. In a preferred aspect, the radiosensitizing agent of the methods of the invention is IPdR.

Nucleoside analogs, such as the halogenated thymidine analogs, IUdR and BUdR, are compounds that “falsely” incorporate into DNA to render cells more susceptible to the lethal effects of radiation therapy by two to three fold, as compared to cells without the defective DNA.

Referring to certain radiosensitizing agents of the invention more specifically, IUdR and IPdR (a prodrug of IUdR) are particularly preferred in the methods of the invention. IUdR (5-iodo-2′-deoxyuridine) is a halogenated thymidine analog that is an effective in vitro and in vivo radiosensitizer. The % IUdR-DNA cellular replacement correlates directly with the extent of radiosensitization. While IUdR has been found to be a potential clinically active radiosensitizer, it requires prolonged continuous intra-arterial or intravenous infusions prior to or during radiation therapy to optimize tumor radiosensitization. However, prolonged continuous infusion presents a challenge in the setting of outpatient radiation therapy and results in myelosuppression and acute GI toxicities that limit the dose and duration of IUdR treatment.

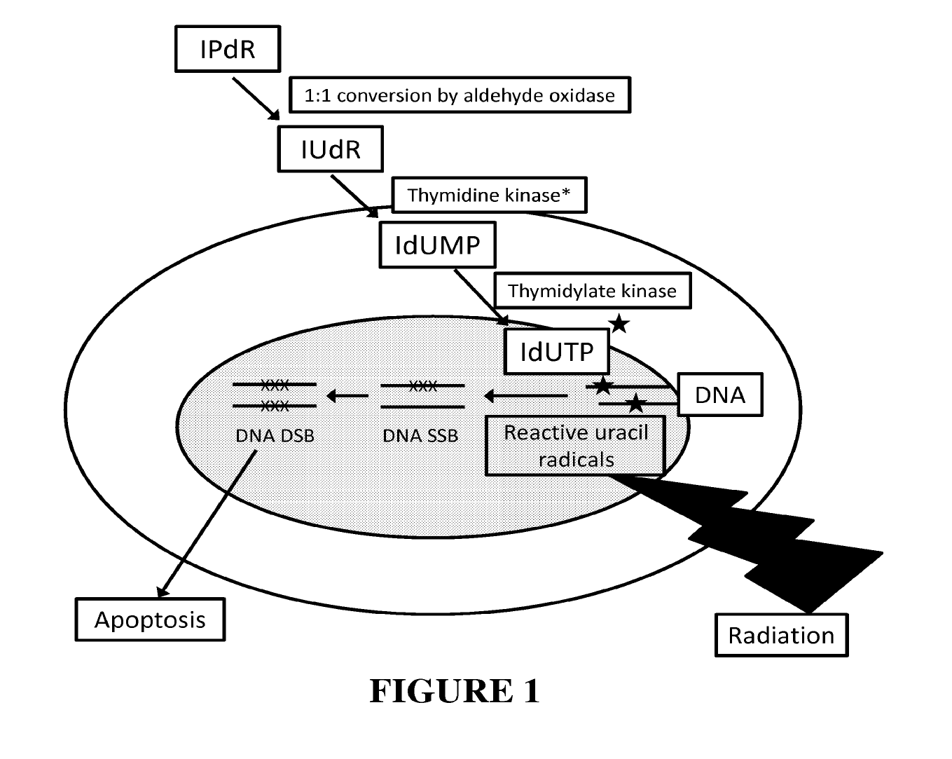

IPdR (5-iodo-2-pyrimidinone-2′-deoxyribose), a prodrug of the radiosensitizer IUdR (5-iodo-2′-deoxyuridine), is orally absorbed and metabolized in the liver to IUdR (FIG. 1). Pre-clinical testing of multiple daily oral IPdR dosing, has demonstrated favorable IPdR to IUdR pharmacokinetics (PK) without significant dose-limiting acute and subacute normal tissue toxicities. Using subcutaneous xenografts of human glioblastoma cell lines and human colorectal cancer cell lines, an improved therapeutic index for radiosensitization has been observed comparing daily oral IPdR to continuous infusion IUdR during radiation therapy. Improved administration and the metabolism of IPdR to IUdR in vivo results in two other major advantages of orally administered (po) IPdR compared to continuous infusion (ci) IUdR. Oral IPdR generates 2-3 fold increased % IUdR-DNA incorporation into tumor tissues and 2-3 fold decreased % IUdR-DNA incorporation into proliferating normal tissues (bone marrow and intestine) as a consequence of the properties and biodistribution of aldehyde oxidase.

Administering the radiosensitizing agent and/or chemotherapeutic agent in the present invention may be accomplished by any means known to a person skilled in the art. The agents used in practicing the method of the invention may be administered in an amount sufficient to induce the desired therapeutic effect in the recipient thereof. Thus the term “therapeutically effective amount” as used herein refers to an amount of the agents which is sufficient to (1) sensitize the cancerous and/or neoplastic cells and tissues to radiation; and/or (2) bring about a detectable therapeutic, preventative, or ameliorative effect (e.g., reduce the quantity of cancerous and/or neoplastic cells). For example, the therapeutically effective amount of a radiosensitizing agent may be that amount that enhances the inhibitory or damaging effect of radiation on cancer cells by at least 10%, at times by at least 20%, 30%, 40%, 50%, 60%, 70% 80%, 90% and even at times by 99-100% of the inhibitory or damaging effect of the radiation on the cancer cells as compared to the effect of radiation of the same cancerous and/or neoplastic cells, without sensitization. Moreover, the magnitude of radiosensitization may be correlated directly to the false incorporation of radiosensitizing agent into suspect DNA. For example, radiosensitization may be correlated directly with the % IUdR-DNA cellular replacement. In fact, the determination of % IUdR-DNA incorporation can serve as a radiosensitization biomarker during treatment.

The radiosensitizing agents of the invention may be administered in one or more doses, at least a portion thereof being given to the patient prior to the patient's exposure to radiation. When a treatment schedule involves administration of several doses of the agent, the doses may be the same or different, for example, escalating or de-escalating amounts per administration. In addition, when referring to a radiosensitizing agent it should be understood as also encompassing a combination of such agents.

The radiosensitizing agents of the present invention are applicable for treating disease in any mammal. Exemplary mammals included laboratory animals, including rodents such as mice, rats and guinea pigs; farm animals such as cows, sheep, pigs and goats; pet animals such as dogs and cats; and primates such as monkeys, apes and humans. The compounds used in the methods of the invention are preferably used in the human treatments.

The method may further include the step of irradiating a selected tissue of the patient before, during, and/or after a radiation sensitizing agent has been administered to the patient. Regarding the application of radiation (i.e., “radiation therapy”) to the patient or subject more generally, such therapy may encompass any ionizing radiation known to those having ordinary skill in the art. Generally, radiation therapy, and in particular ionizing radiation includes applying to a selected tissue, such as a selected tissue comprising cancerous and/or neoplastic cells, a dose of ionizing radiation or two or more fractions of ionizing radiation. The ionizing radiation is defined as an irradiation dose which is determined according to the disease's characteristics at the selected tissue and therapeutic decision of a physician. The term “fractionated dose(s)” may include, for example, conventional fractionation, hyperfractionation, hypofractionation, and accelerated fractionation. The amount of radiation and doses thereof should be sufficient to damage the highly proliferating cells' genetic material, making it impossible for the irradiated cells to continue growing and dividing.

The fractionated irradiation may vary from daily doses (e.g., one or more times per day) given for a period of weeks, or to once weekly doses given for a period of weeks or months. Radiation may be applied in dosages of about 1 Gy to about 100 Gy, or about 20 to about 80 Gy, or more preferably about 30 to 60 Gy.

The dosage in certain embodiments is fractionated, which means that, from about 0.1 to about 10 Gy or from about 1 Gy to about 5 Gy or from about 1 Gy to about 3 Gy are applied in a single session which is repeated several times over the course of about 1 to 10 weeks, or preferably about 2 to 5 weeks. In a preferred aspect of the invention, the radiation dose may be about 30 to 60 Gy at 1 to 3 Gy fractions over a period of about 2 to 5 weeks.

As set forth above, the radiosensitizing agents of the invention may be administered before, after, or together with the radiation. One cycle of radiation therapy as well as several cycles of radiation is possible, dependent of the reduction of tumor size or extent of proliferation. Such sequences of radiosensitization treatments and ionizing irradiation are repeated as needed to abate and, optimally, reduce or eliminate the spread of the cancer or neoplastic cells in the tissue or region of tissue that is selected for treatment. Accordingly, the total dose and the radiation regimen will depend, inter alia, on the cancer type, type of radiosensitizing agent, irradiated area, physical condition of the patient, and many other considerations appreciated by those having ordinary skill in the art.

In addition to the administration of a radiosensitizing agent and the irradiation of the patient, the methods of the invention may include the administration of a therapeutically effective amount of a chemotherapeutic agent to the patient. The chemotherapeutic agent may be provided before, during, or after at least one of the steps of administering the radiosensitizing agent and irradiating a selected tissue of the patient. Therefore, the chemotherapeutic agent may be provided at various points during the methods of the invention for the treatment of disease. In certain aspects, the chemotherapeutic agent may be administered concurrently with or after the step of irradiating the selected tissue of the patient.

The chemotherapeutic agents of the invention may include, but are not limited to, 5-fluorouracil, bevacizumab, capecitabine, topotecan, 6-thioguanine carboplatin, cisplatin, gemcitabine, irinotecan, oxaliplatin, mitomycin-c, paclitaxel, docetaxel, dacarbacine, tamoxifen, etoposide, pemetrexed, cyclophosphamide, doxorubicin, vincristine, amrubicin, an EGFR inhibitor (e.g., cetuximab, erlotinib, gefitinib, lapatinib, panitumumab), temozolomide, and combinations thereof. The chemotherapeutic agent, or its discrete components, where more than one chemotherapeutic agent is combined, may be administered in pure form or together with a pharmaceutically acceptable carrier, lubricant, diluents, excipient, disintegrate, and/or adjuvant mixed together. In preferred embodiments of the invention, the chemotherapeutic agent may be capecitabine, temozolomide, cisplatin, an EGFR inhibitor (e.g., cetuximab, erlotinib, gefitinib, lapatinib, panitumumab), or a combination thereof. In certain aspects of the invention, the chemotherapeutic agents may also be secondary radiosensitizing agents (e.g., capecitabine).

In alternative embodiments, the chemotherapeutic agent may be specifically tailored to the specific cancer or neoplastic disease that is the subject of treatment by the methods of the invention. For example, the chemotherapeutic agents may be used for certain exemplary cancer applications as set forth in Table 1.

TABLE 1
Chemotherapeutics and their Exemplary Cancer Applications.

| Chemotherapeutic | Exemplary Cancer Application |
|---|---|
| 5-Fluorouracil | Pancreatic and Colorectal Cancers |
| Bevacizumab | Colorectal, Breast, Non-Small-Cell |
| | Lung Cancers |
| Capecitabine | Colorectal and Metastatic Breast |
| | Cancers |
| Carboplatin | Head and Neck, Cervical, and |
| | Esophageal Cancers |
| Cetuximab | Head and Neck and Colorectal |
| | Cancers |
| Cisplatin | Cervical, Head and Neck, Colorectal, |
| | and Non-Small-Cell Lung Cancers |
| Gemcitabine | Bladder, Non-Small-Cell Lung, |
| | Pancreatic, Colorectal, and Ovarian |
| | Cancers |
| Irinotecan | Ovarian, Small-Cell, Non-Small- |
| | Cell, Malignant Lymphoma, and |
| | Colorectal Cancers |
| Oxaliplatin | Colorectal and Esophageal Cancers |

In tailoring the methods of the invention to specific disease characteristics in the patient, the methods of the invention may be personalized or patient-specific treatments of cancer where such cancer exhibits a DNA mismatch repair (MMR) deficiency. MMR is a highly conserved, but complex, DNA repair system that helps maintain genomic stability in human cells on several levels, including correcting base-base mismatches and insertion-deletion loops (IDLs) erroneously generated during DNA replication. MMR also mediates cell cycle and cell death in response to certain types of endogenous DNA damage and exogenous DNA damage from occupational and therapeutic chemical and ionizing radiation exposures. As such, MMR plays an essential role in the overall DNA damage response in humans by removing severely damaged cells and reducing the risk of mutagenesis and carcinogenesis. However, in the absence of MMR (i.e., MMR deficiency; MMR⁻), resulting from genetic and/or epigenetic alterations in the human MMR genes, the persistent DNA base-base mismatches and IDLs remaining after DNA replication result in a mutator phenotype with a 10²-10³ elevation of spontaneous mutations highlighted by microsatellite instability (MSI) and a significant risk of cancer.

MMR deficiency is principally associated with the autosomal dominant Lynch Syndrome, a consequence of mutations in MMR genes. MMR deficiency is also associated with an increasing number of sporadic (non-genetic) common solid cancers, typically related to promoter methylation of the hMLH1 and hMSH2 genes. These sporadic MMR deficient human cancers include several types of gastrointestinal cancers (e.g., colorectal, pancreatic, gastric, esophageal), gynecologic cancers (e.g., endometrial, ovarian), genitourinary cancers (bladder, ureter), as well as non-small cell lung cancers (NSCLC) and primary adult brain tumors, where MMR deficiency (detected by standard pathological immunohistochemistry (IHC) testing of MMR protein levels) in these tumors is found in up to 10-20% or more of these common cancers.

In pre-clinical (laboratory based in vitro/in vivo) studies of MMR-deficient human cancer cells, resistance (“damage tolerance”) is found to multiple different classes of clinically active chemotherapy drugs, including temozolomide, topotecan, cisplatinum, carboplatinum, 5-fluorouracil (5-FU), and 6-thioguanine (6-TG), as well as to ionizing radiation.

The clinical implications for the treatment of MMR-deficient sporadic human cancers are of both prognostic and predictive significance. For example, promoter hypermethylation of hMLH1 or hMSH2 with subsequent loss of protein expression of these key MMR regulation proteins by IHC testing is found in nearly 50% of NSCLCs occurring in non-smokers, and is associated with a poor prognosis, even in early stage disease. Additionally, recent analyses of multiple clinical trials of the use of 5-FU (±concomitant cisplatinum or oxaloplatinum) as adjuvant treatment in MMR-deficient colon and esophageal cancers found significantly less benefit in disease-free and overall survival in comparison to a significant benefit in MMR proficient colon and esophageal cancers, respectively. MMR deficient endometrial and rectal cancers also show reduced local control and lower pathological response rates following radiation treatment alone or with combined 5-FU and radiation therapy. Finally, MMR deficient malignant gliomas (high grade adult brain tumors) were noted to have a markedly reduced response rate and survival time compared to MMR proficient gliomas when treated with concomitant radiation therapy and temozolomide.

MMR deficiency occurring during or following cancer treatment may also be associated with a poor prognosis. Somatic point mutations in MSH6 are found in up to 30% of recurrent/progressive glioblastomas, which were not present in pre-treatment specimens. Indeed, inactivation of MSH6 was correlated with prior or ongoing temozolomide exposure and associated with enhanced tumor regrowth and shorter survival. Decreased protein expression of MLH1 following doxorubicin-based chemotherapy in breast cancer patients was also reported to correlate significantly with a reduced disease-free survival (p=0.0025). Finally, promoter methylation of MLH1 in plasma DNA after cisplatin-based chemotherapy for ovarian cancer predicted a poor survival.

Thus, these clinical data underscore the observed resistance (“damage tolerance”) to treatment methods known in the art when comparing pre-clinical studies in MMR deficient and MMR proficient human cancer cells.

As set forth above, IPdR is an orally available, nucleoside analog that is efficiently absorbed in humans and metabolized to IUdR principally in the liver by an aldehyde oxidase. Once metabolized, IUdR is sequentially phosphorylated and competes directly with thymidine for DNA incorporation. While IUdR is an effective radiosensitizing drug, IPdR is a more effective analog based on its oral bioavailability and its improved therapeutic index in pre-clinical (in vivo) human cancer xenograft models. In contrast to other nucleoside analogs, such as 6-TG, the principal DNA mispair (G:IU) resulting from IUdR-DNA incorporation is removed (repaired) by MMR without concomitant cytotoxicity. However, MMR deficient cells treated with IPdR (or IUdR) show higher and prolonged levels of IUdR-DNA incorporation and 2-3 fold greater radiosensitization.

Therefore, in additional aspects, the present invention further includes methods of treating cancers that include cancerous cells having a DNA mismatch repair (MMR) deficiency. Indeed, such treatment methods may function as personalized treatments and may include administering a therapeutically effective amount of a radiosensitizing agent to the patient to sensitize cancerous cells having the MMR deficiency. Moreover, the method may include irradiating a selected tissue of the patient, where the selected tissue includes MMR deficient, sensitized cancerous cells. Further, the method may include the step of providing a therapeutically effective amount of a chemotherapeutic agent to the patient in addition to both the radiosensitizing agent and radiation therapy.

The personalized treatment methods described above may include the step of testing the patient or, more specifically, the cancerous cells of the patient to determine whether such cancerous cells are MMR deficient. If such cells are MMR deficient, the methods of present invention may be utilized to treat the patient exhibiting MMR deficient cancer. As used herein, an “MMR deficient cancer” may include a cancerous tissue and/or cell that substantially lacks MMR protein as detected by standard pathological immunohistochemistry (IHC), a technique known to the person having ordinary skill in the art. Specific MMR deficient cancers may include, for example, gastrointestinal cancers (e.g., colorectal, pancreatic, gastric, esophageal cancers), sarcoma, gynecologic cancers (e.g., endometrial and ovarian cancers), genitourinary cancers (e.g., bladder and ureter cancers), non-small cell lung cancers, and primary adult brain tumors (e.g., malignant gliomas).

Turning to the agents disclosed in the method of the invention more broadly, radiosensitizing agents and/or chemotherapeutic agents may be administered as salts, which is also within the scope of this invention. Pharmaceutically acceptable (i.e., substantially non-toxic, physiologically compatible) salts are preferred. If the agents of the invention have, for example, at least one basic center, they can form acid addition salts. These are formed, for example, with strong inorganic acids, such as mineral acids, for example sulfuric acid, phosphoric acid or a hydrohalic acid, with strong organic carboxylic acids, such as alkane carboxylic acids of 1 to 4 carbon atoms which are unsubstituted or substituted, for example, by halogen, for example acetic acid, such as saturated or unsaturated dicarboxylic acids, for example oxalic, malonic, succinic, maleic, fumaric, phthalic or terephthalic acid, such as hydroxycarboxylic acids, for example ascorbic, glycolic, lactic, malic, tartaric or citric acid, such as amino acids, (for example aspartic or glutamic acid or lysine or arginine), or benzoic acid, or with organic sulfonic acids, such as (C₁-C₄) alkyl or arylsulfonic acids which are unsubstituted or substituted, for example by halogen, for example methyl- or para-toluene-sulfonic acid. Corresponding acid addition salts can also be formed having plural basic centers, if desired. The agents of the invention having at least one acid group (for example COOH) can also form salts with suitable bases. Representative examples of such salts include metal salts, such as alkali metal or alkaline earth metal salts, for example sodium, potassium or magnesium salts, or salts with ammonia or an organic amine, such as morpholine, thiomorpholine, piperidine, pyrrolidine, a mono, di or tri-lower alkylamine, for example ethyl, tert-butyl, diethyl, diisopropyl, triethyl, tributyl or dimethyl-propylamine, or a mono, di or trihydroxy lower alkylamine, for example mono, di or triethanolamine. Corresponding internal salts may also be formed.

All stereoisomers of the agents disclosed herein existing either in a mixture or in pure or substantially pure form, are considered to be within the scope of this invention. The agents of the present invention can have asymmetric centers at any of the carbon atoms including any one of the substituents. Consequently, agents used in the method of the invention can exist in enantiomeric or diastereomeric forms or in mixtures thereof. The processes for preparation of such compounds can utilize racemates, enantiomers or diastereomers as starting materials. When diastereomeric or enantiomeric products are prepared, they can be separated by conventional methods for example, chromatographic, chiral HPLC, fractional crystallization or distillation. Some compounds of the present invention have groups including alkenyls, iminyls, and the like, which may exist as entgegen (E) or zusammen (Z) conformations, in which case all geometric forms thereof, both E and Z, cis and trans, and mixtures thereof, are within the scope of the present invention. Accordingly, when such geometric isomeric products are prepared, they can be separated by conventional methods for example, chromatographic, HPLC, distillation or crystallization.

The agents utilized in the method of the invention may be administered as such, or in a form from which the active agent can be derived, such as a prodrug. A “prodrug” is a derivative of a compound described herein, the pharmacologic action of which results from the conversion by chemical or metabolic processes in vivo to the active compound. For instance, as set forth herein, IPdR is a prodrug of IUdR. Prodrugs may include compounds wherein an amino acid residue, or a polypeptide chain of two or more (e.g., two, three or four) amino acid residues is covalently joined through an amide or ester bond to a free amino, hydroxyl or carboxylic acid group of the radiosensitizing agent or chemotherapeutic agent. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Prodrug esters as employed herein includes esters and carbonates formed by reacting one or more hydroxyls of compounds of the method of the invention with alkyl, alkoxy, or aryl substituted acylating agents employing procedures known to persons having ordinary skill in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., IPdR to IUdR) is a prodrug within the scope and spirit of the invention.

The radiosensitizing agents and/or chemotherapeutic agents used in the methods of the invention described herein may be administered at a dose in a range from about 0.01 mg/M² to about 5000 mg/M². A dose of from 0.1 to 3000 mg/M², and preferably from 100 to 2000 mg/M² in one or more applications per day may be effective to produce the desired result. For example, radiosensitizing agents of the invention may be administered once or twice daily at a dose in a range of about 0.01 to 3000 mg/M². In a preferred aspect, radiosensitizing agents of the invention (e.g., IPdR) may be administered at a dose in a range from about 1500 to 2000 mg/M². Of course, as those skilled in the art will appreciate, the dosage actually administered will depend upon the condition being treated, the age, health and weight of the recipient, the type of concurrent treatment, if any, and the frequency of treatment. Moreover, the effective dosage amount may be determined by one skilled in the art on the basis of routine empirical activity testing to measure the bioactivity of the agents in a bioassay (either in vitro or in vivo), and thus establish the appropriate dosage to be administered.

In general, the agents used in the methods of the invention can be administered to provide radiosensitization as set forth above using any acceptable route known in the art, either alone or in combination with one or more other therapeutic agents. Thus, the agent(s) of the invention can be administered orally, parenterally, such as by intravenous or intraarterial infusion, intramuscular, intraperitoneal, intrathecal or subcutaneous injection, by liposome-mediated delivery, rectally, vaginally, by inhalation or insufflation, transdermally or by otic delivery.

The orally administered dosage unit may be in the form of tablets, caplets, dragees, pills, semisolids, soft or hard gelatin capsules, aqueous or oily solutions, emulsions, suspensions or syrups. Suitable dosage forms for parenteral administration include injectable solutions or suspensions, suppositories, powder formulations, such as microcrystals or aerosol spray. The active agents of the invention may also be incorporated into a conventional transdermal delivery system.

As used herein, the expression “physiologically compatible carrier medium” includes any and all solvents, diluents, or other liquid vehicle, dispersion or suspension aids, surface agent agents, isotonic agents, thickening or emulsifying agents, preservatives, solid binders, lubricants, fillers and the like as suited for the particular dosage form desired. Remington: The Science and Practice of Pharmacy, 20^{th} edition, A. R. Genaro et al., Part 5, Pharmaceutical Manufacturing, pp. 669-1015 (Lippincott Williams & Wilkins, Baltimore, Md./Philadelphia, Pa.) (2000) discloses various carriers used in formulating pharmaceutical compositions and known techniques for the preparation thereof. Except insofar as any conventional pharmaceutical carrier medium is incompatible with either the radiosensitizing or chemotherapeutic compounds used in the present invention, such as by producing an undesirable biological effect or otherwise interacting in an deleterious manner with any other component(s) of a formulation comprising such compounds or agents, its use is contemplated to be within the scope of this invention.

For the production of solid dosage forms, including hard and soft capsules, the agents of the invention may be mixed with pharmaceutically inert, inorganic or organic excipients, such as lactose, sucrose, glucose, gelatine, malt, silica gel, starch or derivatives thereof, talc, stearic acid or its salts, dried skim milk, vegetable, petroleum, animal or synthetic oils, wax, fat, polyols, and the like. For the production of liquid solutions, emulsions or suspensions or syrups one may use excipients such as water, alcohols, aqueous saline, aqueous dextrose, polyols, glycerine, lipids, phospholipids, cyclodextrins, vegetable, petroleum, animal or synthetic oils. For suppositories one may use excipients, such as vegetable, petroleum, animal or synthetic oils, wax, fat and polyols. For aerosol formulations, one may use compressed gases suitable for this purpose, such as oxygen, nitrogen and carbon dioxide. Pharmaceutical compositions or formulations may also contain one or more additives including, without limitation, preservatives, stabilizers, e.g., UV stabilizers, emulsifiers, sweeteners, salts to adjust the osmotic pressure, buffers, coating materials and antioxidants.

The present invention further includes controlled-release, sustained-release, or extended-release therapeutic dosage forms for administration of the agents of the invention, which involves incorporation of the agents into a suitable delivery system. This dosage form controls release of the active agent(s) in such a manner that an effective concentration of the active agent(s) in the bloodstream may be maintained over an extended period of time, with the concentration in the blood remaining relatively constant, to improve therapeutic results and/or minimize side effects. Additionally, a controlled-release system would provide minimum peak to trough fluctuations in blood plasma levels of the agent.

In pharmaceutical compositions used in practicing the method of the invention, the specified agent(s) may be present in an amount of at least 0.5 and generally not more than 95% by weight, based on the total weight of the composition, including carrier medium and/or supplemental active agent(s), if any. Preferably, the proportion of active agent(s) varies between 30-90% by weight of the composition.

The methods of the invention may further include the step of surgically resecting a cancerous tissue where the cancerous tissue includes a solid tumor. Therefore, resection of the cancerous tissue may be performed before or after at least one of the steps of administering a therapeutically effective amount of a radiosensitizing agent, applying radiation therapy to the patient, and providing a therapeutically effective amount of a chemotherapeutic agent to the patient. As used herein, resection of the solid tumor may allow for the mass to be reduced prior to application of the methods of the invention.

In additional aspects of the methods of the invention, such methods may be used as second line methods of treatment for patients where such patients were provided with a standard therapy that failed. For example, cisplatin is a first line treatment for head and neck cancers. However, in certain instances, the patient may not respond to cisplatin or simply relapse after a certain period of time. In such instances where the patient relapses, the cancer or neoplastic disorder can be more difficult to treat. The present method can thus provide a second line method of treatment after certain first line methodologies fail or are inadequate. Examples of first line treatments may be found in Table 1. Alternatively, the method of the invention may also be used as a third line method of treatment.

In accordance with the foregoing methods of the invention, a kit is provided that may include a radiosensitizing agent (one or more of such agents), a chemotherapeutic agent (one or more of such agents), and instructions for use of the radiosensitizing agent and the chemotherapeutic agent in combination with radiation therapy for treating cancer or neoplastic disease in a patient in need of such treatment.

The it according to present invention may be used in the met as described herein.

As set forth herein, radiation sensitization allows for improvement in the results of cancer radiation therapy by affecting cellular processes to achieve greater levels of cancer cell killing by the delivered radiation. The goal of an ideal radiation sensitizer has not been achieved. Generally, chemotherapeutic agents and biologics with single agent activities, as well as associated radiation sensitizing capacity, are used in combination with radiation therapy (Table 1). The drawback of this approach lies in drug dose limitations and drug related toxicities. For example, known chemotherapeutic agents exhibit a variety of toxicities, such as nausea, vomiting, myelosuprresion, enteritis, oral mucositis, impaired wound healing, hypertension, bleeding problems, increased risk of thromboemolic events, CNS neurotoxicity, diarrhea, hand/foot syndrome, myelosuppression, neuropathy, nephrotoxicity, dermatitis, pulmonary fibrosis, hypomagnesemia, infusional allergic reactions, gastrointestinal toxicity, peripheral neuropathy, asthenia, ototoxicity, anorexia/weight loss, and fatigue.

Pre-operative chemoradiation, using either continuous infusion 5-fluorouracil (5-FU) or twice daily capecitabine, is currently the standard of care for patients with clinical Stage 2 and 3 rectal cancers. However, only about 45% of patients achieve tumor (T stage) downstaging, and less than 20% of patients achieve a pathologic complete response (pCR) at the time of surgery, defined as no viable tumor cells found in the resected specimen. The extent of tumor downstaging, and particularly pCR, is associated with improved local control, overall and cancer specific-survival; as well as allowing for sparing of the rectal sphincter in some patients where an abdomino-perineal resection would otherwise have been necessary. In spite of the development of new systemic agents for patients with advanced (metastatic) colorectal cancer (e.g. oxaliplatin, irinotecan, bevacizumab, and EGFR inhibitors), none have increased the pCR rate in locally advanced rectal cancer patients when used with infusional 5-FU and radiation therapy, but they have increased normal tissues toxicities. In contrast, capecitabine administered twice daily is equivalent to infusional 5-FU in terms of pCR, local recurrence, and overall survival.

Capecitabine is an orally available fluoropyrimidine carbamate that generates the active drug, 5-FU, selectively in tumors, by three enzymes located in the liver and in tumors. The final step is the conversion of the intermediate metabolite, 5′dFUrd into 5-FUra by thymidine phosphorylase (dThdPase) in tumors. Without being limited by any one theory, this conversion seems to be a rate-limiting step for capecitabine efficacy. Toxicities of capecitabine include hand-foot syndrome, oral mucositis, and diarrhea.

While the precise molecular/biochemical mechanisms of fluoropyrimidine-mediated radiosensitization of rectal tumors are not fully understood, and without being limited to any one theory of the invention, radiosensitization is principally mediated by 5-FU effects on the production and balance of intracellular DNA precursor (nucleotide) pools via binding of its metabolite, FdUMP to thymidylate synthase (TS) causing TS inhibition and subsequent depletion of thymidine triphosphate (dTTP) pools. The level of TS expression in colorectal cancer cell lines, as well in patient tumor specimens, is of prognostic significance with high TS expression correlating with reduced radiosensitization experimentally and in patients.

The extent of human tumor radiosensitization by IUdR or its prodrug, IPdR, is directly correlated with the % IUdR-DNA incorporation. Biochemically, the common intracellular metabolite of IUdR and IPdR, i.e. IdUTP, competes with dTTP for DNA incorporation and, as such, any strategy to selectively decrease intracellular dTTP pools in human tumors will increase the % IUdR-DNA incorporation and IUdR-mediated radiosensitization. Experimentally, 5-FU/FdUrD and/or leucovorin increase % IUdR-DNA incorporation and IUdR-mediated radiosensitization via reduced TS activity (greater inhibition) using human colorectal cancer cell lines. However, by combining continuous intravenous infusions of IUdR with either continuous infusions of FdUrd or leucovorin, biochemical modulation of % IUdR-DNA incorporation could not clearly be demonstrated using circulating granulocytes from patients as surrogates of tumor cells.

Given the greater tumor selectivity for TS inhibition and radiosensitization by capecitabine in rectal cancer patients compared to continuous infusion 5-FU, and given the extensive pre-clinical data showing a 2-3 fold increase in % IUdR-DNA incorporation in tumors and a 2 fold decrease in % IUdR/DNA in normal bone marrow and gut epithelium by using daily oral IPdR compared to continuous infusion IUdR, the concomitant daily use of oral capecitabine and oral IPdR during radiation therapy in patients with cancer, such as locally advanced rectal cancer, may significantly enhance tumor radiosensitization resulting in a higher pCR rate (>30%) with acceptable normal tissue toxicities. This may, in turn, correlate with increased survival and improved quality of life for these patients. By establishing the MTD and documenting the plasma PK data for once daily oral administration of IPdR combined with fractionated radiation therapy (37.5 Gy @ 2.5 Gy fractions over 3 weeks), the daily (or twice daily) IPdR dose may be reduced by two dose levels, while using standard doses of capecitabine (750 mg/M²) and radiation therapy (50.4-54 Gy @ 1.8 Gy fractions).

The following examples describe the invention in further detail. These examples are provided for illustrative purposes only, and should in no way be considered as limiting the invention.

### EXAMPLES

### Example 1: Efficacy of IUdR as a Radiosensitizing Agent Compared to Historical RT-Alone Controls for Treatment of High Grade Primary Brain Tumors

IUdR requires continuous IV infusion—not an ideal potential candidate for clinical use. Nevertheless, the administration of IUdR as a radiosensitizer in patients exhibiting gliomas led to increased overall survival (Table 2).

TABLE 2

| | | Median Survival |
|---|---|---|
| Tumor | Treatment | (Months) |
| Anaplastic Astroyctomas | RT alone | 24 |
| (Grade 3 of 4) | RT + IUdR | 39 |
| Glioblastoma Multiforme | RT alone | 9 |
| (Grade 4 of 4) | RT + IUdR | 15 |

### Example 2: Efficacy of IUdR as a Radiosensitizing Agent Compared to Historical RT-Alone Controls for Treatment of High Grade Sarcomas

The administration of IUdR as a radiosensitizer in patients exhibiting high grade sarcomas demonstrated significant delay in disease progression for both resectable and un-resectable sarcomas (Table 3).

TABLE 3

| | | Median Survival |
|---|---|---|
| Tumor | Treatment | (Months) |
| High Grade Sarcomas | RT + Surgery | 25% |
| (Resectable) | RT + IUdR + Surgery | 45% |
| High Grade Sarcomas | RT alone | <10% |
| (Un-Resectable) | RT + IUdR | 60% |

### Example 3: Treatment of Locally Advanced High Grade Sarcomas and Rectal Cancer

The methods of the invention may be used to treat both advanced sarcomas and rectal cancer.

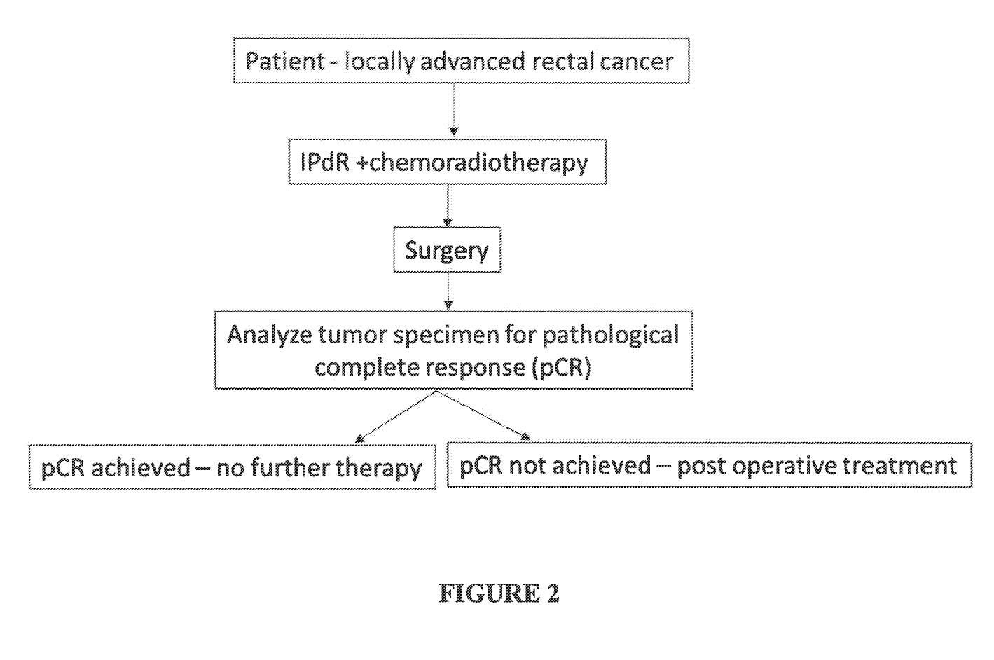

For example, as diagrammed in FIG. 2, where a patient is diagnosed with rectal cancer, a dose of IPdR can be orally administered to the patient to sensitize cancerous cells to radiation. Following sensitization, the patient will then be treated with chemoradiotherapy (i.e., radiation in combination with a chemotherapeutic agent such as capecitabine). After sensitization and chemoradiotherapy, any solid tumors can be surgically resected or removed, if possible. After treatment, a tumor specimen will be extracted and analyzed for pathological complete response (pCR).

Where pCR is achieved, no further therapy is required. If pCR is not achieved, post operative treatment is required and will include additional chemotherapy or treatment with IPdR and radiotherapy. Additional IPdR therapy can also be followed by the administration of chemotherapeutic agents.

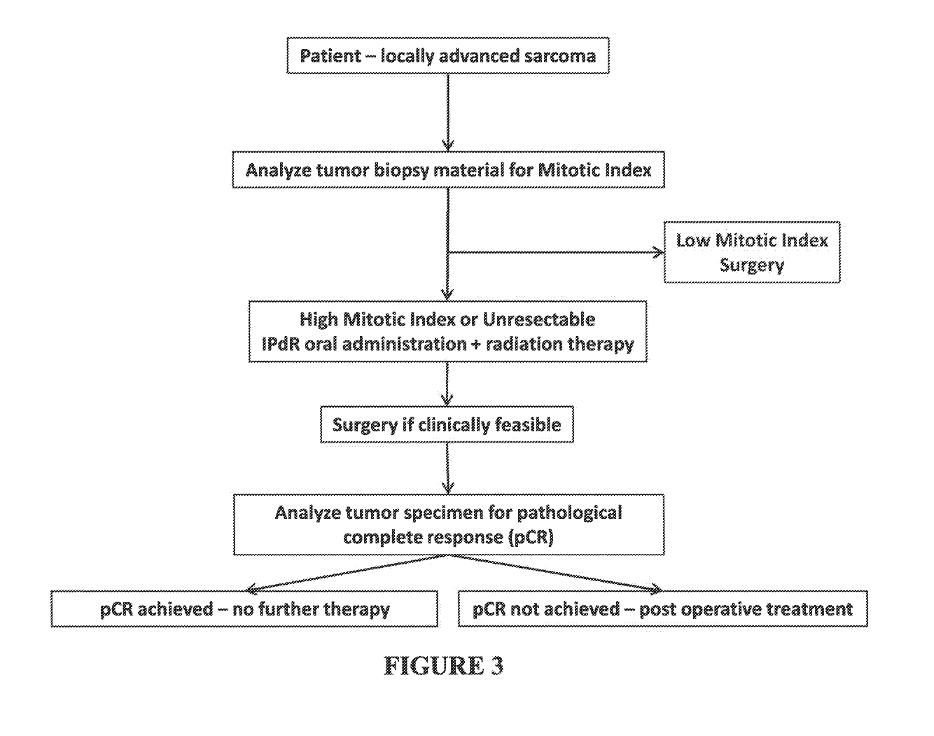

An additional exemplary method is diagrammed in FIG. 3. Where a patient presents with a locally advanced sarcoma, a solid tumor will be biopsied to extract tissue for analysis. The extracted tumor biopsy material will then be analyzed for its mitotic index. Where the mitotic index is low, the solid tumor will be surgically removed. However, if the mitotic index is high, or the solid tumor is not resectable, the patient will receive an oral dose of IPdR in combination with radiation therapy. The administration of IPdR and radiation therapy may be repeated as necessary to shrink any solid tumors. At this stage, if clinically feasible, the solid tumors will be surgically removed or the patient will be treated with an additional chemotherapeutic agent (e.g., cisplatin).

Following treatment, a tumor specimen will be extracted and analyzed for pCR. Where pCR is achieved, no further therapy is required. If pCR is not achieved, post operative treatment is required and will include additional chemotherapy or treatment with IPdR and radiotherapy. Additional IPdR therapy can also be followed by the administration of chemotherapeutic agents.

### Example 4: Treatment of Locally Advanced Rectal Cancer Having a Deficient Mismatch Repair (MMR) Pathway

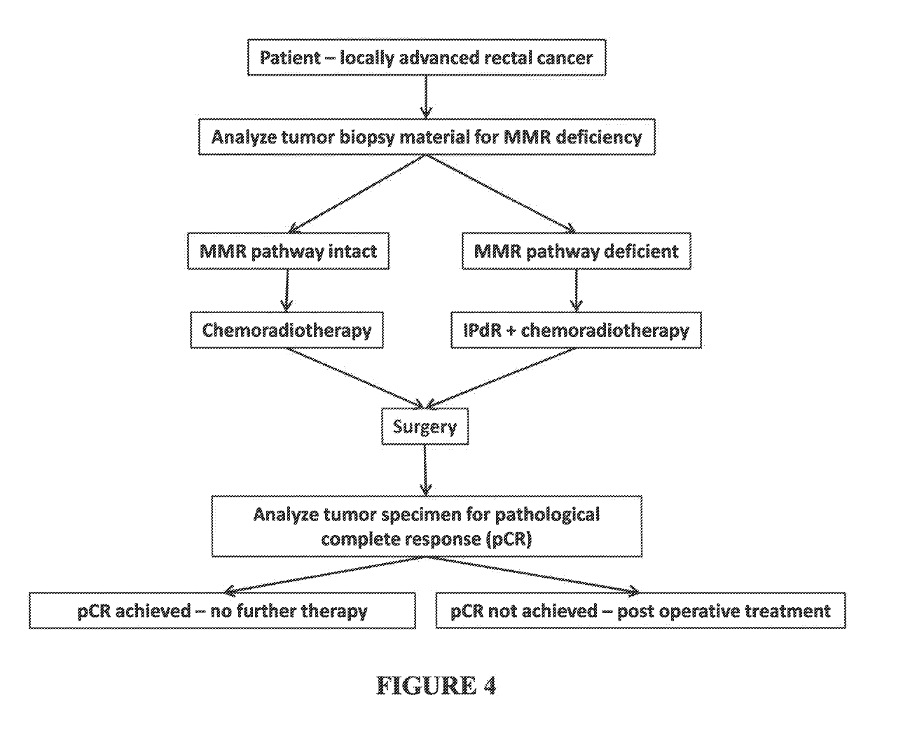

Methods of the invention can be used to treat locally advanced rectal cancer, where such cancer may or may not have an intact mismatch repair (MMR) pathway (FIG. 4).

Where the patient presents with a locally advanced rectal cancer, a solid tumor can be biopsied to extract tissue for analysis. The extracted tissue biopsy material will then be analyzed for MMR deficiency. Where the MMR pathway is intact, the patient will be treated with chemoradiotherapy followed by surgical resection of the solid tumor. Chemoradiotherapy includes radiation in combination with a chemotherapeutic agent such as capecitabine. Where the MMR pathway is deficient, a dose of IPdR will be orally administered to the patient followed by chemoradiotherapy and surgical resection of the solid tumor.

Following surgical resection of the solid tumor, in both the MMR intact and MMR deficient cases, a tumor specimen will be extracted and analyzed for pCR. Where pCR is achieved, no further therapy is required. If pCR is not achieved, post operative treatment is required and will include additional chemotherapy or treatment with IPdR and chemoradiotherapy.

A number of patent and non-patent publications are cited herein in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All methods, kits, and other embodiments described herein that embody the present invention can, in alternate embodiments, be more specifically defined by any of the transitional terms “comprising,” “consisting essentially of,” and “consisting of.”

### REFERENCES

1. Lin S H, George T J, Ben-Josef E, et al; Participants on Workshop for Preclinical and Clinical Development of Radiosensitizers; National Cancer Institute. Opportunities and challenges in the era of molecularly targeted agents and radiation therapy. J Natl Cancer Inst. 2013 May 15; 105(10):686-93. doi: 10.1093/jnci/djt055. Epub 2013 Mar. 15. PMID: 23503600
2. Kinsella T J. An approach to the radiosensitization of human tumors. The cancer journal from Scientific American. July-August 1996; 2(4):184-193.
3. Cook J A, Glass J, Lebovics R, et al. Measurement of thymidine replacement in patients with high grade gliomas, head and neck tumors, and high grade sarcomas after continuous intravenous infusions of 5-iododeoxyuridine. Cancer research. Feb. 1, 1992; 52(3):719-725.
4. Epstein A H, Lebovics R S, Goffman T, et al. Treatment of locally advanced cancer of the head and neck with 5′-iododeoxyuridine and hyperfractionated radiation therapy: measurement of cell labeling and thymidine replacement. Journal of the National Cancer Institute. Dec. 7, 1994; 86(23):1775-1780.
5. Knol J A, Walker S C, Robertson J M, et al. Incorporation of 5-bromo-2′-deoxyuridine into colorectal liver metastases and liver in patients receiving a 7-day hepatic arterial infusion. Cancer research. Sep. 1, 1995; 55(17):3687-3691.
6. Chang C N, Doong S L, Cheng Y C. Conversion of 5-iodo-2-pyrimidinone-2′-deoxyribose to 5-iodo-deoxyuridine by aldehyde oxidase. Implication in hepatotropic drug design. Biochem Pharmacol. May 28, 1992; 43(10):2269-2273.
7. Kinsella T J, Kunugi K A, Vielhuber K A, McCulloch W, Liu S H, Cheng Y C. An in vivo comparison of oral 5-iodo-2′-deoxyuridine and 5-iodo-2-pyrimidinone-2′-deoxyribose toxicity, pharmacokinetics, and DNA incorporation in athymic mouse tissues and the human colon cancer xenograft, HCT-116. Cancer research. May 15, 1994; 54(10):2695-2700.
8. Kinsella T J, Kunugi K A, Vielhuber K A, Potter D M, Fitzsimmons M E, Collins J M. Preclinical evaluation of 5-iodo-2-pyrimidinone-2′-deoxyribose as a prodrug for 5-iodo-2′-deoxyuridine-mediated radiosensitization in mouse and human tissues. Clin Cancer Res. January 1998; 4(1):99-109.
9. Kinsella T J, Vielhuber K A, Kunugi K A, Schupp J, Davis T W, Sands H. Preclinical toxicity and efficacy study of a 14-day schedule of oral 5-iodo-2-pyrimidinone-2′-deoxyribose as a prodrug for 5-iodo-2′-deoxyuridine radiosensitization in U251 human glioblastoma xenografts. Clin Cancer Res. April 2000; 6(4):1468-1475.
10. Kinsella T J, Kinsella M T, Seo Y, Berk G. 5-iodo-2-pyrimidinone-2′-deoxyribose-mediated cytotoxicity and radiosensitization in U87 human glioblastoma xenografts. International journal of radiation oncology, biology, physics. Nov. 15, 2007; 69(4):1254-1261.
11. Sauer R, Becker H, Hohenberger W, et al. Preoperative versus postoperative chemoradiotherapy for rectal cancer. N Engl J Med. Oct. 21, 2004; 351(17):1731-1740.
12. Krishnamurthi S S, Seo Y, Kinsella T J. Adjuvant therapy for rectal cancer. Clin Colon Rectal Surg. August 2007; 20(3): 167-181.
13. Martin S T, Heneghan H M, Winter D C. Systematic review and meta-analysis of outcomes following pathological complete response to neoadjuvant chemoradiotherapy for rectal cancer. Br J Surg. July 2012; 99(7):918-928.
14. Park I J, You Y N, Agarwal A, et al. Neoadjuvant treatment response as an early response indicator for patients with rectal cancer. J Clin Oncol. May 20, 2012; 30(15):1770-1776.
15. Dipetrillo T, Pricolo V, Lagares-Garcia J, et al. Neoadjuvant bevacizumab, oxaliplatin, 5-fluorouracil, and radiation for rectal cancer. International journal of radiation oncology, biology, physics. Jan. 1, 2012; 82(1):124-129.
16. Hofheinz R D, Wenz F, Post S, et al. Chemoradiotherapy with capecitabine versus fluorouracil for locally advanced rectal cancer: a randomised, multicentre, non-inferiority, phase 3 trial. Lancet Oncol. June 2012; 13(6):579-588.
17. Miller E M, Kinsella T J. Radiosensitization by fluorodeoxyuridine: effects of thymidylate synthase inhibition and cell synchronization. Cancer research. Apr. 1, 1992; 52(7):1687-1694.
18. Bruso C E, Shewach D S, Lawrence T S. Fluorodeoxyuridine-induced radiosensitization and inhibition of DNA double strand break repair in human colon cancer cells. International journal of radiation oncology, biology, physics. December 1990; 19(6):1411-1417.
19. Saif M W, Berk G, Cheng Y C, Kinsella T J. IPdR: a novel oral radiosensitizer. Expert Opin Investig Drugs. September 2007; 16(9):1415-1424.
20. Lawrence T S, Davis M A, Maybaum J, Stetson P L, Ensminger W D. Modulation of iododeoxyuridine-mediated radiosensitization by 5-fluorouracil in human colon cancer cells. International journal of radiation oncology, biology, physics. 1992; 22(3):499-503.
21. Miller E M, Kunugi K A, Kinsella T J. Effects of 5′-aminothymidine and leucovorin on radiosensitization by iododeoxyuridine in human colon cancer cells. Clin Cancer Res. April 1995; 1(4):407-416.
22. Speth P A, Kinsella T J, Belanger K, et al. Fluorodeoxyuridine modulation of the incorporation of iododeoxyuridine into DNA of granulocytes: a phase I and clinical pharmacological study. Cancer research. May 15, 1988; 48(10):2933-2937.
23. McGinn C J, Kunugi K A, Tutsch K D, et al. Leucovorin modulation of 5-iododeoxyuridine radiosensitization: a phase I study. Clin Cancer Res. August 1996; 2(8):1299-1305.
25. Cheema A K, Varghese R S, Timofeeva O, et al, Functional proteomics analysis to study ATM dependent signaling in response to ionizing radiation. Radiat Res. 2013 June; 179(6):674-83. doi: 10.1667/RR3198.1. Epub 2013 May 3. PMID: 2364204
26 Cheema A K, Timofeeva O, Varghese R, et al. Integrated analysis of ATM mediated gene and protein expression impacting cellular metabolism. J Proteome Res. 2011 May 6; 10(5):2651-7. doi: 10.1021/pr101243j. Epub 2011 Mar. 7. PMID: 21322649
27. Kinsella T J, Kinsella M T, Hong S, Johnson J P, Burback B, Tosca P J. Toxicology and pharmacokinetic study of orally administered 5-iodo-2-pyrimidinone-2′deoxyribose (IPdR)×28 days in Fischer-344 rats: impact on the initial clinical phase I trial design of IPdR-mediated radiosensitization. Cancer Chemother Pharmacol. February 2008; 61(2): 323-334.
28. Kinsella T J, Schupp J E, Davis T W, et al. Preclinical study of the systemic toxicity and pharmacokinetics of 5-iodo-2-deoxypyrimidinone-2′-deoxyribose as a radiosensitizing prodrug in two, non-rodent animal species: implications for phase I study design. Clin Cancer Res. September 2000; 6(9):3670-3679.
29. Seo Y, Yan T, Schupp J E, Radivoyevitch T, Kinsella T J. Schedule-dependent drug effects of oral 5-iodo-2-pyrimidinone-2′-deoxyribose as an in vivo radiosensitizer in U251 human glioblastoma xenografts. Clin Cancer Res. Oct. 15, 2005; 11(20):7499-7507.
30. Kummar S, Anderson L, Hill K, et al. First-in-human phase 0 trial of oral 5-iodo-2-pyrimidinone-2′-deoxyribose in patients with advanced malignancies. Clin Cancer Res. Apr. 1, 2013; 19(7):1852-1857.
31. Dupertuis Y M, Vazquez M, Mach J P, et al. Fluorodeoxyuridine improves imaging of human glioblastoma xenografts with radiolabeled iododeoxyuridine. Cancer research. Nov. 1, 2001; 61(21):7971-7977.
32. Iyer R R, Pluciennik A, Burdett V, Modrich P L. DNA mismatch repair: functions and mechanisms. Chem Rev. 2006; 106(2):302-23.
33. Jiricny J. The multifaceted mismatch-repair system. Nat Rev Mol Cell Biol. 2006; 7(5):335-46.
34. Li G M. Mechanisms and functions of DNA mismatch repair. Cell Res. 2008; 18(1):85-98.
35. Peltomaki P. Role of DNA mismatch repair defects in the pathogenesis of human cancer. J Clin Oncol. 2003; 21(6):1174-9.
36. Kinsella T J. DNA Mismatch Repair: It's role in human carcinogenesis and as a predictive and/or prognostic biomarker for cancer therapy. In: Madhusudan S, Wilson, D. M., editor. DNA Repair and Cancer: Bench to Clinic Enfield, N.H.: Science Publishers; 2013. p. 195-232.
37. Lynch H T, de la Chapelle A. Hereditary colorectal cancer. N Engl J Med.

2003; 348(10):919-32.
38. Karran P. Mechanisms of tolerance to DNA damaging therapeutic drugs. Carcinogenesis. 2001; 22(12): 1931-7.
39. Kinsella T J. Coordination of DNA mismatch repair and base excision repair processing of chemotherapy and radiation damage for targeting resistant cancers. Clin Cancer Res. 2009; 15(6):1853-9.
40. Brown K D, Rathi A, Kamath R, Beardsley D I, Zhan Q, Mannino J L, et al. The mismatch repair system is required for S-phase checkpoint activation. Nat Genet. 2003; 33(1):80-4.
41. Yan T, Seo Y, Kinsella T J. Differential cellular responses to prolonged LDR-IR in MLH1-proficient and MLH1-deficient colorectal cancer HCT116 cells. Clin Cancer Res. 2009; 15(22):6912-20. PMCID: 2783277.
42. Hsu H S, Wen C K, Tang Y A, Lin R K, Li W Y, Hsu W H, et al. Promoter hypermethylation is the predominant mechanism in hMLH1 and hMSH2 deregulation and is a poor prognostic factor in nonsmoking lung cancer. Clin Cancer Res. 2005; 11(15):5410-6.
43. Kishi K, Doki Y, Yano M, Yasuda T, Fujiwara Y, Takiguchi S, et al. Reduced MLH1 expression after chemotherapy is an indicator for poor prognosis in esophageal cancers. Clin Cancer Res. 2003; 9(12):4368-75.
44. Sargent D J, Marsoni S, Monges G, Thibodeau S N, Labianca R, Hamilton S R, et al. Defective mismatch repair as a predictive marker for lack of efficacy of fluorouracil-based adjuvant therapy in colon cancer. J Clin Oncol. 2010; 28(20):3219-26. PMCID: 2903323.
45. Bilbao C, Lara P C, Ramirez R, Henriquez-Hernandez L A, Rodriguez G, Falcon O, et al. Microsatellite instability predicts clinical outcome in radiation-treated endometrioid endometrial cancer. Int J Radiat Oncol Biol Phys. 2010; 76(1):9-13. PMCID: 2796581.
46. Choi M Y, Lauwers G Y, Hur C, Willett C G, Chung D C. Microsatellite instability is frequently observed in rectal cancer and influenced by neoadjuvant chemoradiation. Int J Radiat Oncol Biol Phys. 2007; 68(5):1584.
47. Friedman H S, McLendon R E, Kerby T, Dugan M, Bigner S H, Henry A J, et al. DNA mismatch repair and O6-alkylguanine-DNA alkyltransferase analysis and response to Temodal in newly diagnosed malignant glioma. J Clin Oncol. 1998; 16(12):3851-7.
48. Cahill D P, Levine K K, Betensky R A, Codd P J, Romany C A, Reavie L B, et al. Loss of the mismatch repair protein MSH6 in human glioblastomas is associated with tumor progression during temozolomide treatment. Clin Cancer Res. 2007; 13(7):2038-45. PMCID: 2873832.
49. Mackay H J, Cameron D, Rahilly M, Mackean M J, Paul J, Kaye S B, et al. Reduced MLH1 expression in breast tumors after primary chemotherapy predicts disease-free survival. J Clin Oncol. 2000; 18(1):87-93.
50. Gifford G, Paul J, Vasey P A, Kaye S B, Brown R. The acquisition of hMLH1 methylation in plasma DNA after chemotherapy predicts poor survival for ovarian cancer patients. Clin Cancer Res. 2004; 10(13):4420-6.
51. Berry S E, Kinsella T J. Targeting DNA mismatch repair for radiosensitization. Semin Radiat Oncol. 2001; 11(4):300-15.
52. Berry S E, Loh T, Yan T, Kinsella T J. Role of MutSalpha in the recognition of iododeoxyuridine in DNA. Cancer Res. 2003; 63(17):5490-5.
53. Seo Y, Yan T, Schupp J E, Colussi V, Taylor K L, Kinsella T J. Differential radiosensitization in DNA mismatch repair-proficient and -deficient human colon cancer xenografts with 5-iodo-2-pyrimidinone-2′-deoxyribose. Clin Cancer Res. 2004; 10(22): 7520-8.

1. A method for treating cancer in a patient in need of such treatment, the method comprising:
a. administering a therapeutically effective amount of a radiosensitizing agent to the patient to sensitize cancerous cells to radiation, wherein the radiosensitizing agent comprises a halogenated nucleoside;
b. irradiating a selected tissue of the patient, wherein the selected tissue comprises sensitized cancerous cells; and
c. providing a therapeutically effective amount of a chemotherapeutic agent to the patient.

2. The method of claim 1, wherein the halogenated nucleoside comprises a halogenated thymidine analog or a pharmaceutically acceptable salt thereof.

3. The method of claim 1, wherein the halogenated nucleoside comprises 5-iodo-2-pyrimidinone-2′-deoxyribose (IPdR), 5-iodo-2′-doxyuridine (IUdR), 5-bromo-2′-deoxyuridine (BUdR), or a pharmaceutically acceptable salt thereof.

4. The method of claim 1, wherein the halogenated nucleoside comprises 5-iodo-2-pyrimidinone-2′-deoxyribose (IPdR) or a pharmaceutically acceptable salt thereof.

5. The method of claim 1, wherein the cancer comprises pancreatic cancer, colorectal cancer, breast cancer, gastric cancer, non-small-cell lung cancer, metastatic breast cancer, head and neck cancers, endometrial cancer, ovarian cancer, ureter cancer, cervical cancer, esophageal cancer, bladder cancer, ovarian cancer, small-cell cancer, non-small cell cancer, malignant lymphomas, brain cancer, rectal cancer, sarcomas, or a combination thereof.

6. The method of claim 1, wherein the cancer is at least one of rectal cancer and brain cancer.

7. The method of claim 1, wherein the step of irradiating a selected tissue of the patient comprises providing about 1 Gy to 100 Gy of radiation.

8. The method of claim 1, wherein the step of irradiating a selected tissue of the patient comprises providing about 20 Gy to 80 Gy of radiation.

9. The method of claim 1, wherein the step of irradiating a selected tissue of the patient comprises providing about 30 Gy to 60 Gy of radiation.

10. The method of claim 1, wherein the step of irradiating a selected tissue of the patient comprises providing a fractionated dose of radiation to the patient.

11. The method according to claim 10, wherein the fractionated dose comprises about 0.1 Gy to about 10 Gy of radiation.

12. The method of claim 1, wherein the chemotherapeutic agent comprises 5-fluorouracil, bevacizumab, capecitabine, carboplatin, cisplatin, gemcitabine, irinotecan, oxaliplatin, topotecan, 6-thioguanine, mitomycin-c, paclitaxel, docetaxel, dacarbacine, tamoxifen, etoposide, pemetrexed, cyclophosphamide, doxorubicin, vincristine, amrubicin, an EGFR inhibitor, temozolomide, or a combination thereof.

13. The method according to claim 12, wherein the EGFR inhibitor comprises cetuximab, erlotinib, gefitinib, lapatinib, panitumumab, or a combination thereof.

14. The method of claim 1, wherein the chemotherapeutic agent comprises capecitabine, temozolomide, an EGFR inhibitor, or a combination thereof.

15. The method of claim 1, wherein the step of providing a therapeutically effective amount of a chemotherapeutic agent comprises providing a dose of the chemotherapeutic agent in a range from about 0.01 mg/M² to about 5000 mg/M².

16. The method of claim 1, wherein the step of providing a therapeutically effective amount of a chemotherapeutic agent comprises providing a dose of the chemotherapeutic agent in a range from about 0.1 mg/M² to about 3000 mg/M².

17. The method of claim 1, wherein the step of providing a therapeutically effective amount of a chemotherapeutic agent comprises providing a dose of the chemotherapeutic agent in a range from about 100 mg/M² to about 2000 mg/M².

18. The method of claim 1, wherein the step of providing a therapeutically effective amount of a chemotherapeutic agent to the patient occurs concomitantly with at least one of the steps of administering and irradiating.

19. The method of claim 1, wherein the step of providing a therapeutically effective amount of a chemotherapeutic agent to the patient occurs after at least one of the steps of administering and irradiating.

20. The method of claim 1, wherein the step of providing a therapeutically effective amount of a chemotherapeutic agent to the patient occurs before at least one of the steps of administering and irradiating.

21. The method of claim 1, wherein the method is a second line method of treatment for the patient and the step of administering occurs after performance of a first line therapy on the patient that failed to treat the cancer.

22. The method of claim 1, wherein the method is a third line method of treatment for the patient and the step of administering occurs after performance of a second line therapy on the patient that failed to treat the cancer.

23. The method of claim 1, wherein the selected tissue comprises a surgically resected selected tissue.

24. The method of claim 1, the method further comprising the step of repeating steps a, b, and c.

25. The method of claim 1, wherein at least one of the radiosensitizing agent and the chemotherapeutic agent comprise a physiologically compatible carrier medium.

26. The method of claim 1, wherein the step of administering a therapeutically effective amount of a radiosensitizing agent comprises a route of administration selected from the group consisting of oral, parenteral, liposome-mediated, rectal, vaginal, inhalation, insufflations, transdermal, otic administration, and combinations thereof.

27. The method of claim 1, wherein the step of providing a therapeutically effective amount of a chemotherapeutic agent comprises a route of administration selected from the group consisting of oral, parenteral, liposome-mediated, rectal, vaginal, inhalation, insufflations, transdermal, otic administration, and combinations thereof.

28. A kit for providing a method for treating cancer in a patient in need of such treatment, the kit comprising a radiosensitizing agent, a chemotherapeutic agent, and instructions for use of the radiosensitizing agent and the chemotherapeutic agent in combination with radiation therapy for treating cancer in the patient in need of such treatment.

29. The kit of claim 28, wherein the radiosensitizing agent comprises 5-iodo-2-pyrimidinone-2′-deoxyribose (IPdR), 5-iodo-2′-doxyuridine (IUdR), 5-bromo-2′-deoxyuridine (BUdR), or a pharmaceutically acceptable salt thereof.

30. The kit of claim 28, wherein the chemotherapeutic agent comprises 5-fluorouracil, bevacizumab, capecitabine, carboplatin, topotecan, 6-thioguanine, cisplatin, gemcitabine, irinotecan, oxaliplatin, mitomycin-c, paclitaxel, docetaxel, dacarbacine, tamoxifen, etoposide, pemetrexed, cyclophosphamide, doxorubicin, vincristine, amrubicin, an EGFR inhibitor, temozolomide, or a combination thereof.

31. The kit of claim 28, wherein the cancer to be treated is selected from the group consisting of pancreatic cancer, colorectal cancer, breast cancer, gastric cancer, non-small-cell lung cancer, metastatic breast cancer, head and neck cancers, endometrial cancer, ovarian cancer, ureter cancer, cervical cancer, esophageal cancer, bladder cancer, ovarian cancer, small-cell cancer, non-small cell cancer, malignant lymphomas, brain cancer, rectal cancer, sarcoma, and combinations thereof.

32. A method for treating a DNA mismatch repair (MMR) deficient cancer in a patient in need of such treatment where cancer cells of the patient exhibit a lack of MMR protein as compared to non-cancerous cells, said DNA MMR deficient cancer being selected from the group consisting of esophageal cancer, endometrial cancer, bladder cancer, non-small cell lung cancer, and brain cancer, the method comprising the steps of:
a. administering a radiosensitizing agent to the patient to sensitize the cancer cells to radiation;
b. irradiating the cancer cells that have been sensitized to radiation; and
c. administering at least one chemotherapeutic agent to the patient;
wherein the radiosensitizing agent and the at least one chemotherapeutic agent are administered in therapeutically effective amounts sufficient to sensitize the cancer cells to radiation or reduce the quantity of cancer cells in the patient.

33. The method according to claim 32, wherein the radiosensitizing agent comprises 5-iodo-2-pyrimidinone-2′-deoxyribose (IPdR), 5-iodo-2′-doxyuridine (IUdR), 5-bromo-2′-deoxyuridine (BUdR), or a pharmaceutically acceptable salt thereof.

34. The method of claim 32, wherein the chemotherapeutic agent comprises 5-fluorouracil, bevacizumab, capecitabine, carboplatin, cisplatin, gemcitabine, irinotecan, oxaliplatin, topotecan, 6-thioguanine, mitomycin-c, paclitaxel, docetaxel, dacarbacine, tamoxifen, etoposide, pemetrexed, cyclophosphamide, doxorubicin, vincristine, amrubicin, an EGFR inhibitor, temozolomide, or a combination thereof.

35. A method for treating cancer in a patient in need of such treatment, wherein the cancer is selected from the group consisting of esophageal cancer, endometrial cancer, bladder cancer, non-small cell lung cancer, and brain cancer, the method comprising:
a. determining whether the patient has DNA mismatch repair (MMR) protein deficient cancer by retrieving at least one cancer cell from the patient and testing the at least one cancer cell to determine the level of MMR protein;
b. administering to a patient determined to have MMR protein deficient cancer a therapeutically effective amount of a radiosensitizing agent to sensitize cancerous cells to radiation; wherein the sensitized cancerous cells are MMR deficient;
c. irradiating a selected tissue of the patient, wherein the selected tissue comprises sensitized, MMR deficient cancerous cells; and
d. providing a therapeutically effective amount of a chemotherapeutic agent to the patient.
